(19) United States
(12) Patent Application Publication
(10) Pub. No.: US 2017/0267608 A1
(43) Pub. Date: Sep. 21, 2017

(54) SINTERING-RESISTANT NANOSIZED IRON OXIDE BASED CATALYSTS

(71) Applicant: SABIC Global Technologies B.V., Bergen op Zoom (NL)

(72) Inventors: Heli WANG, Sugar Land, TX (US); Ihab N. ODEH, Sugar Land, TX (US)

(21) Appl. No.: 15/610,827

(22) Filed: Jun. 1, 2017

Related U.S. Application Data
(60) Provisional application No. 62/348,008, filed on Jun. 9, 2016.

(51) Int. Cl. C07C 5/333; B01J 35/00; B01J 35/02; B01J 23/745; B01J 37/08

(52) U.S. Cl. CPC C07C 5/3332; B01J 23/745; B01J 37/08; B01J 35/023; B01J 35/0013; C07C 2523/745

(57) ABSTRACT

Disclosed is a catalyst and methods to prepare and use the catalyst in an ethylbenzene dehydration reaction. The catalyst contains α-Fe₂O₃ and a dopant(s) and/or a promoter(s), has a size of 2 nanometers to 50 nanometers, and does not include γ-Fe₂O₃. The catalyst can be a bulk catalyst or can be supported by a ceramic or other appropriate support material.

### CROSS REFERENCE TO RELATED APPLICATIONS

This application claims benefit to U.S. Provisional Application No. 62/348,008 titled “Sintering-resistant Nanosized Iron Oxide Based Catalysts”, filed Jun. 9, 2016, which is incorporated herein by reference in its entirety.

### BACKGROUND OF THE INVENTION

A. Field of the Invention

The invention concerns a nanostructured α-phase hematite-based catalyst and methods for preparing and using the catalyst in hydrocarbon dehydrogenation reactions. The catalyst can have an average size of 2 to 50 nanometers and can include dopants and/or promoters. The catalyst can be completely devoid of the maghemite (γ-Fe₂O₃) crystalline phase.

B. Description of Related Art

Hematite-based iron (III) oxide (Fe₂O₃) catalysts—due to low cost and ease of preparation—have been used for a variety of hydrocarbon dehydrogenation reactions. Dehydrogenation plays a major role in the production of styrene, for example, accounting for 90%, or roughly 20 million tons per year, of worldwide production of the compound. The ethylbenzene to styrene dehydrogenation reaction is illustrated below:

C₆H₅—CH₂—CH₃→C₆H₅—CH═CH₂+H₂.

Styrene is an important building block for a variety of polymers such as latex, synthetic rubber, and polystyrene resins. These polymers can be used to produce various plastic products, such as packaging, disposable containers, insulation, and others.

Fe₂O₃ exists in a number of crystalline phases: α-; β-; γ-; and εFe₂O₃. The β- and ε-Fe₂O₃ phases are metastable, and convert to the α-Fe₂O₃ phase at moderate temperatures. While it has long been used for styrene production via dehydrogenation of ethylbenzene, Fe₂O₃ catalysts nevertheless suffer a number of inherent drawbacks, most notably overall catalytic activity. This problem is particularly seen with pure α-Fe₂O₃ catalysts, which tend to have relatively large particle sizes and therefore reduced surface area and ultimately reduced catalytic activity. By way of example, B. Xiang, et al., Reaction Kinetics Catalysis Letters, Vol. 94, No. 1, (2008) 175-182, discloses a pure α-Fe₂O₃ phase catalyst with an average feature size of 98 nm. The catalyst, however, had poor catalytic activity for the ethylbenzene to styrene dehydrogenation reaction when compared to a mixed α- and γ-Fe₂O₃ phase catalyst having a feature size of 19 nm.

As illustrated in Xiang et al., one of the challenges facing pure α-Fe₂O₃ catalysts is the controlled production of small nanostructures (i.e., on the order of a few tens-of-nanometers at most). This challenge is likely caused by two primary reasons: (1) Ostwald ripening; and (2) sintering. Ostwald ripening has the effect of producing nanostructures with larger average features sizes when the nanomaterial is processed at higher relative temperatures. Therefore, given that α-Fe₂O₃ forms at temperatures higher than does γ-Fe₂O₃, nanomaterials of the former are expected to be larger (see Xiang et al.). In addition to these larger sizes, pure α-Fe₂O₃ catalysts also exhibit sintering when subjected to the aforementioned higher temperatures.

While attempts have been made to reduce particle size and sintering, the Ostwald effect nevertheless persists for pure α-Fe₂O₃ catalysts and the need for smaller nanostructures (with feature sizes on the order of a few tens of nanometers at most) remains.

### SUMMARY OF THE INVENTION

A solution to the above-mentioned problems associated with α-Fe₂O₃ catalysts has been discovered The solution resides in the production of an α-phase hematite-based catalyst that includes an α-Fe₂O₃ crystalline phase but is free of/does not include a γ-Fe₂O₃ crystalline phase. The size of the catalyst is relatively small (e.g., 2 to 50 nanometers, preferably 5 to 40 nanometers, more preferably 10 to 30 nanometers, or even more preferably 10 nanometers to 25 nanometers). In particular, the catalysts of the present invention provide a solution to the aforementioned Ostwald ripening and sintering issues plaguing the production of α-Fe₂O₃ catalysts that have relatively larger sizes (greater than 50 nm). It has been discovered that a staged calcination process can be used to eliminate the γ-Fe₂O₃ phase, leaving pure α-Fe₂O₃ nanostructures with a characteristic feature size less than 50 nm in a single- or poly-crystalline form. These nanostructures can also be loaded with a promoter(s) and/or a dopant(s) affording additional resistance to sintering. The catalyst of the present invention can exhibit improved performance in hydrocarbon dehydration reactions as compared to conventional Fe₂O₃ catalysts. Without wishing to be bound by theory, it is believed that while α-Fe₂O₃ and γ-Fe₂O₃ phases are both catalytically active, α-Fe₂O₃ is the desired phase with regards to dehydrogenation reactions. The preference for the hematite phase may be attributed to absence of crystallographic vacancies in the rhombohedral hematite crystal lattice that are otherwise present in the cubic maghemite lattice. Lack of vacancies in the hematite phase may manifest in enhanced α-Fe₂O₃ activity relative to γ-Fe₂O₃, caused by higher lattice energy (and therefore structural stability), improved charge transport (facilitating electron flow in the course of the catalytic reaction), and more uniform exposure of catalytically-active Fe³⁺ sites. Therefore, the present invention provides for an α-phase hematite-based catalyst having a characteristic feature size less than 50 nm that has good catalytic performance for hydrocarbon dehydrogenation reactions such as the ethylbenzene to styrene reaction.

In a particular aspect of the invention, there is disclosed a nanostructured catalyst comprised of iron (III) oxide existing in the hematite (α-Fe₂O₃) phase along with a promotor and/or dopant. Multiple promoters and/or dopants can be loaded into the catalyst as well. Further, absent from the catalysts of the present invention is the maghemite (γ-Fe₂O₃) phase. In other more particular embodiments, the catalysts can also exclude the β- and ε-Fe₂O₃) phases. The nanostructured α-phase hematite-based catalyst of the present invention can have a general formula of [FeₓA_{y}B_{z}]Oₙ, wherein A is a dopant, B is a promoter, x is 1−(y+z), 0≦y≦0.10, 0≦z≦0.30, n is determined by valence requirements of Fe and A, B, or the sum of A and B, with the caveat that y or z is greater than 0. In some embodiments, the dopant and/or promoter can be included in the lattice structure of the α-Fe₂O₃ crystalline phase. In other embodiments, the dopant and/or promoter can be more localized at the surface or bulk of the catalyst. Non-limiting examples of dopants and promoters are provided throughout the specification and claims and incorporated into this paragraph by reference. In some particularly preferred embodiments, the promoter can be potassium oxide (K₂O).

The nanostructured catalysts of the present invention can have a high surface area-to-volume ratio, which is achieved through reduction of the overall nanostructure size (i.e., less than 50 nm). As such, the catalysts of the present invention can have an average size of 2 nanometers to 50 nanometers, 5 nanometers to 40 nanometers, 10 nanometers and 30 nanometers, or 10 nanometers and 25 nanometers. In preferred instances, a size of 10 to 30 nanometers or 10 to 25 nanometers can be used.

The nanostructured catalysts of the present invention can be prepared through a staged thermal treatment process. This can include obtaining a composition comprising iron containing precursor material and dopant(s) and/or promoter(s) solubilized in the composition (e.g., aqueous solution or organic solution). Iron-containing precursor material can include iron salts, organometallic iron compounds, iron-containing complexed compounds, or any other suitable precursor material. The dopant(s) and/or promoter(s) can be salts, organometallics, complexes, or other appropriate compounds. In even more particular embodiments, ligand compound(s) can be added to the reaction medium to help control the growth rate and size of the nanostructure particles. Appropriate ligands may comprise organic compounds from the following classes: alkenes, alkynes, alcohols, aldehydes, carboxylic acids, phosphines, phosphonates, phosphonic acids, sulfates, sulfides, amines, or any other appropriate class of compound. The composition can then be subjected to a temperature of 80° C. to 200° C. for 30 minutes to 24 hours, preferably 90° C. to 180° C. for 30 minutes to 24 hours. The resulting dried material can then optionally be rinsed (e.g., with an aqueous solution or an organic solution). The dried material can then be thermally treated at 400° C. to 600° C. in the presence of an oxygen source for 30 minutes to 4 hours. This can be followed by an additional thermal treatment at a temperature of 750° C. to 850° C. for 30 minutes to 4 hours to obtain the nanostructured α-phase hematite catalyst. The additional thermal treatment can be performed in the presence of air or other oxygen sources.

The nanostructured catalysts of the present invention, prepared as described above, can be used for hydrocarbon dehydrogenation reactions under conditions sufficient to dehydrogenate the hydrocarbon. In one embodiment, the reaction is dehydrogenation of ethylbenzene to styrene, where the catalyst is in contact with a reactant feed that includes ethylbenzene at a reaction temperature of 500° C. to 700° C. Pressure used in the reaction can range from about 0.01 MPa to about 2.0 MPa. The gas hourly space velocity (GHSV) of the reactant feed can range from 100 h⁻¹ to 100,000 h⁻¹ or more. In the course of such a reaction, the catalyst may be attached to a support structure. Appropriate support materials include ceramic oxides (SiO₂ and Al₂O₃ serve as non-limiting examples) or nanotubes (including, but not limited to carbon nanotubes or boron nitride nanotubes).

In a particular instance, 20 embodiments are described. Embodiment 1 is a nanostructured α-phase hematite-based catalyst capable of catalyzing a dehydrogenation of hydrocarbon reaction, the catalyst comprising an α-Fe₂O₃ crystalline phase and a dopant(s) and/or a promoter(s), wherein the catalyst has a size of 2 nanometers to 50 nanometers and does not include a γ-Fe₂O₃ crystalline phase. Embodiment 2 is the nanostructured α-phase hematite-based catalyst of embodiment 1, wherein the catalyst is a single- or poly-crystalline α-Fe₂O₃ phase catalyst. Embodiment 3 is the nanostructured α-phase hematite-based catalyst of embodiment 1, wherein the catalyst does not include a magnetite (Fe₃O₄) crystalline phase. Embodiment 4 is the nanostructured α-phase hematite-based catalyst of any one of embodiments 1 to 3, wherein the catalyst has a general structure of:

[FeₓA_{y}B_{z}]Oₙ,

wherein A is a dopant, B is a promoter, x is 1−(y+z), 0≦y≦0.10, 0≦z≦0.30, n is determined by valence requirements of Fe and A and/or B, with the proviso that y or z is greater than 0. Embodiment 5 is the nanostructured α-phase hematite-based catalyst of any one of embodiment 1 to 4, wherein the dopant(s) and/or promoter(s) is/are comprised in the lattice structure of the α-Fe₂O₃ crystalline phase. Embodiment 6 is the nanostructured α-phase hematite-based catalyst of any one of embodiments 1 to 5, having a size of 2 nanometers to 40 nanometers, 5 nanometers to 30 nanometers, or 10 nanometers to 25 nanometers. Embodiment 7 is the nanostructured α-phase hematite-based catalyst of any one of embodiments 1 to 6, wherein the catalyst comprises a dopant, the dopant comprising antimony (Sb), arsenic (As), bismuth (Bi), silicon (Si), germanium (Ge), tin (Sn), lead (Pb), aluminium (Al), gallium (Ga), indium (In), thallium (Tl), titanium (Ti), vanadium (V), chromium (Cr), molybdeum (Mo), manganese (Mn), platinum (Pt), lanthanum (La), cerium (Ce), or any combination, or oxide, or alloy thereof. Embodiment 8 is the nanostructured α-phase hematite-based catalyst of any one of embodiments 1 to 7, wherein the catalyst comprises a promoter, the promoter comprising an alkali metal, an alkali earth metal, a transition metal, or a lanthanoid, or any combination, or oxide, or alloy thereof selected from Lithium (Li), Sodium (Na), Potassium (K), Rubidium (Rb), Cesium (Cs), Beryllium (Be), Magnesium (Mg), Calcium (Ca), Strontium (Sr), Barium (Ba), Scandium (Sc), Yttrium (Y), Zirconium (Zr), Hafnium (Hf), Vanadium (V), Niobium (Nb), Tantalum (Ta), Chromium (Cr), Molybdenum (Mo), Tungsten (W), Manganese (Mn), Technetium (Tc), Rhenium (Re), Ruthenium (Ru), Osmium (Os), Cobalt (Co), Rhodium (Rh), Iridium (Ir), Nickel (Ni), Palladium (Pd), Platinum (Pt), Copper (Cu), Silver (Ag), Gold (Au), Zinc (Zn), Cadmium (Cd), Mercury (Hg), Aluminum (Al), Gallium (Ga), Indium (In), Thallium (Tl), Germanium (Ge), Lead (Pb), Lanthanum (La), Cerium (Ce) Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (TM), Ytterbium (Yb), or Lutetium (Lu). Embodiment 9 is the nanostructured α-hematite-based catalyst of any one of embodiments 1 to 8, wherein the catalyst is the product of a hydrothermal reaction of (i) a composition comprising precursors of iron and the dopant and/or promoter with (ii) thermal treatment, wherein the thermal treatment comprises subjecting the composition to a temperature of 80° C. to 200° C. for 30 minutes to 24 hours, followed by a temperature of 400° C. to 600° C. for 30 minutes to 4 hours, followed by a temperature of 750° C. to 850° C. for 30 minutes to 4 hours. Embodiment 10 is the nanostructured α-hematite-based catalyst of any one of embodiments 1 to 9, wherein the catalyst is a bulk catalyst having an average nanostructure size of 2 nanometers to 50 nanometers, 2 nanometers to 40 nanometers, 5 nanometers to 30 nanometers, or 10 nanometers to 25 nanometers. Embodiment 11 is the nanostructured α-hematite-based catalyst of any one of embodiments 1 to 10, wherein the catalyst is sinter and/or coke resistant during use. Embodiment 12 is the nanostructured α-hematite-based catalyst of any one of embodiments 1 to 11, wherein the dehydrogenation of hydrocarbon reaction is dehydrogenation of ethylbenzene to styrene. Embodiment 13 is the nanostructured α-hematite-based catalyst of embodiment 12, wherein the catalyst is in contact with a reactant feed that includes ethylbenzene. Embodiment 14 is the nanostructured α-hematite-based catalyst of embodiment 13, wherein the reactant feed has a temperature of 500° C. to 700° C. Embodiment 15 is a method of dehydrogenating a hydrocarbon, the method comprising contacting a reactant feed that includes a hydrocarbon with any one of the nanostructured α-phase hematite-based catalysts of embodiment 1 to 14 under conditions sufficient to dehydrogenate the hydrocarbon. Embodiment 16 is the method of claim 15, wherein the hydrocarbon in the reactant feed is ethylbenzene, and wherein the ethylbenzene is dehydrogenated to styrene. Embodiment 17 is the method of embodiment 16, wherein the reactant feed has a temperature of 500° C. to 700° C.

Embodiment 18 is a method of making a nanostructured α-phase hematite-based catalyst of any one of embodiments 1 to 14, the method comprising: (a) obtaining an aqueous solution comprising an iron containing precursor material and a dopant(s) and/or a promoter(s), wherein each of the precursor material and the dopant(s) and/or promoter(s) are solubilized in the aqueous solution; (b) heating the aqueous solution to obtain a crystalline material comprising iron and dopant(s) and/or promoter(s), wherein the crystalline material has an average size of 2 nanometers to 50 nanometers; and (c) subjecting the crystalline material from step (b) to a temperature of 400° C. to 600° C. for 30 minutes to 4 hours in the presence of an oxygen source followed by a temperature of 750° C. to 850° C. for 30 minutes to 4 hours to obtain the nanostructured α-phase hematite catalyst of any one of embodiments 1 to 14. Embodiment 19 is the method of embodiment 18, wherein the iron containing precursor material is an iron salt. Embodiment 20 is the method of any one of embodiments 18 to 19, wherein heating step (b) comprises heating the aqueous solution to 80 to 200° C., preferably 90° C. to 180° C., for 30 minutes to 24 hours

The following includes definitions of various terms and phrases used throughout this specification.

The term “catalyst” means a substance which alters the rate of a chemical reaction. “Catalytic” means having the properties of a catalyst.

The term “conversion” means the mole fraction (i.e., percent) of a reactant converted to a product or products.

The term “selectivity” refers to the percent of converted reactant that went to a specified product, e.g., styrene selectivity is the % of ethylbenzene that formed styrene (as opposed to toluene, for example).

“Nanostructure” means a structure having features in at least one dimension with a size on the order of 2 nanometers to 50 nanometers. In a particular aspect, the nanostructure includes at least two dimensions with a size of 2 nanometers to 50 nanometers. In another aspect, the nanostructure includes all three dimensions with a size of 2 nanometers to 50 nanometers. The shape of the nanostructure can be of a wire, a particle (e.g., having a substantially spherical shape), a rod, a tetrapod, a hyper-branched structure, a tube, a cube, or mixtures thereof.

The terms “about” or “approximately” are defined to be within 10%, preferably within 5%, more preferably within 1%, and most preferably within 0.5%.

The term “substantially” refers to ranges within 10%, within 5%, within 1%, or within 0.5%.

The terms “inhibiting” or “reducing” or “preventing” or “avoiding” or any variation of these terms, when used in the claims and/or the specification, include any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The terms “wt. %,” “vol. %,” or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the nanostructured catalysts of the present invention is the exclusion of the γ-Fe₂O₃ phase while having a nanostructured size between 2 and 50 nanometers.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is an illustration of a single crystal nanoparticle catalyst of the present invention.

FIG. 2 is an illustration of a polycrystalline nanoparticle catalyst of the present invention.

FIG. 3 is a schematic of a system for using the nanostructured catalyst of the present invention in a hydrocarbon dehydrogenation reaction (e.g., ethylbenzene to styrene).

### DETAILED DESCRIPTION OF THE INVENTION

Currently available catalysts for dehydrogenation reactions such as ethylbenzene to styrene often suffer from reduced activity and gradual deactivation. These effects may be caused by particle sintering, which reduces the area of active material that would otherwise be available to catalyze the dehydrogenation reaction, and/or coking, which has a similar effect, but whose cause is carbonaceous deposition on the surface of the catalyst.

A discovery has been made that provides a solution to the currently available catalysts. In particular, an α-phase hematite-based nanostructured catalyst having an average size of 2 to 50 nanometers has been discovered. The catalyst includes an α-Fe₂O₃ crystalline phase along with a promotor and/or dopant but is free of/does not include a γ-Fe₂O₃ crystalline phase. Such a catalyst can have higher ethylbenzene (C₆H₅—CH₂—CH₃)-to-styrene (C₆H₅—CH═CH₂) conversion and higher C₆H₅—CH═CH₂ selectivity (relative to byproducts that include, but are not limited to, toluene and benzene) than commercially available iron oxide-based catalysts. The catalyst can also have reduced sintering and/or coking during use.

These and other non-limiting aspects of the present invention are discussed in further detail in the following sections.

A. α-Fe₂O₃ Catalyst

The Fe₂O₃ catalysts of the present invention include catalytic Fe₂O₃ nanostructures and may include a support material. The catalysts can further include a promotor or multiple promoters, which serves to inhibit coking and/or sintering of the catalysts at high temperatures and/or pressures. The catalysts can also include a dopant or multiple dopants, which serves to improve the electrical conductivity through the catalyst to increase catalytic activity.

1. α-Fe₂O₃ Material

The nanostructured α-phase hematite-based catalysts of the present invention can have a general formula [FeₓA_{y}B_{z}]Oₙ, wherein A is a dopant, B is a promoter, x is 1−(y+z), 0≦y≦0.10, 0≦z≦0.30, n is determined by valence requirements of Fe and A, Fe and B, or Fe, A, and B, with the caveat that y or z is greater than 0. More specifically, the catalytic material can include the α-phase (hematite) iron (III) oxide (α-Fe₂O₃). Though unlikely to occur (i.e., given that both phases are metastable and convert to α-Fe₂O₃ at temperatures above 500° C.), the catalysts may also contain β-Fe₂O₃ and/or ε-Fe₂O₃. Further, the catalysts may or may not contain magnetite (Fe₃O₄). The γ-Fe₂O₃ phase (maghemite), however, can be absent from the catalysts of the present invention. Non-limiting examples of a hematite-based catalyst of the present invention can be a powdered material having varying mesh sizes, including nanosized α-Fe₂O₃ particles or structures, or combinations thereof. The catalyst nanostructures can also be loaded with a dopant(s) (A) and/or a promoter(s) (B). The catalyst can also contain a plurality of different promoters and/or different dopants.

2. Promoters

Promoters can provide resistance against sintering and coking. The effects of sinter- and coke-resistance manifest in extended catalyst lifetime, as a larger portion of catalytically-active surface area remains exposed to the reactant fluid for a longer period of time than it otherwise would if no promoter were present in the catalyst. Non-limiting examples of promoters that can be used with the catalysts of the present invention include materials having one or more elements comprising Lithium (Li), Sodium (Na), Potassium (K), Rubidium (Rb), Cesium (Cs), Beryllium (Be), Magnesium (Mg), Calcium (Ca), Strontium (Sr), Barium (Ba), Scandium (Sc), Yttrium (Y), Zirconium (Zr), Hafnium (Hf), Vanadium (V), Niobium (Nb), Tantalum (Ta), Chromium (Cr), Molybdenum (Mo), Tungsten (W), Manganese (Mn), Technetium (Tc), Rhenium (Re), Ruthenium (Ru), Osmium (Os), Cobalt (Co), Rhodium (Rh), Iridium (Ir), Nickel (Ni), Palladium (Pd), Platinum (Pt), Copper (Cu), Silver (Ag), Gold (Au), Zinc (Zn), Cadmium (Cd), Mercury (Hg), Aluminum (Al), Gallium (Ga), Indium (In), Thallium (Tl), Germanium (Ge), Lead (Pb), Lanthanum (La), Cerium (Ce) Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (TM), Ytterbium (Yb), Lutetium (Lu), or any combination, or oxide, or salt, or alloy or other suitable form thereof

3. Dopants

Dopants can improve electrical conductivity within the catalysts of the present invention, thereby improving charge transport and promoting the homogenous reaction at the hematite surface. Non-limiting examples of dopant that can be used with the catalysts of the present invention include materials having one or more elements comprising antimony (Sb), arsenic (As), bismuth (Bi), silicon (Si), germanium (Ge), tin (Sn), lead (Pb), aluminium (Al), gallium (Ga), indium (In), thallium (Tl), titanium (Ti), vanadium (V), chromium (Cr), molybdeum (Mo), manganese (Mn), platinum (Pt), lanthanum (La), cerium (Ce), or any combination, or oxide, or salt, or alloy or other suitable form thereof.

4. Crystallinity of the Catalyst

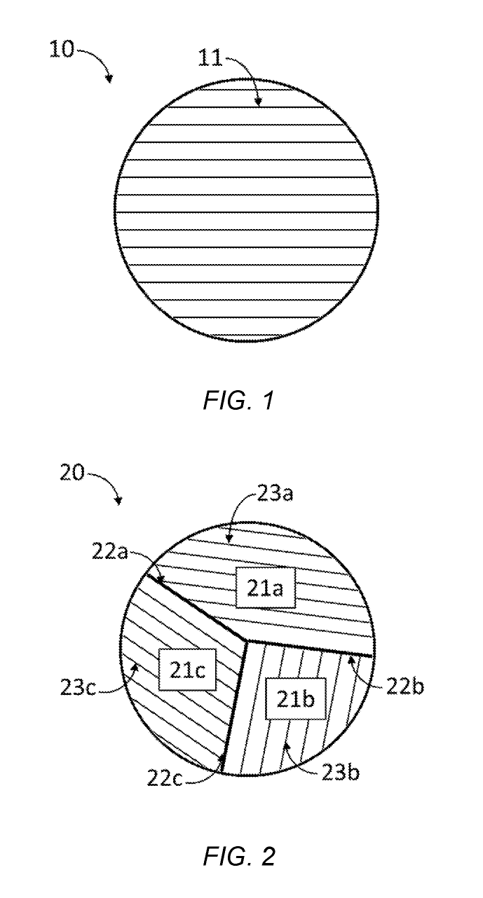

Electrical conductivity of the catalysts of the present invention can be tuned or modified as desired through controlling the crystallinity of the catalyst material. The catalyst nanostructures (e.g, nanoparticles) can be single crystalline or polycrystalline. Referring to FIG. 1, a single crystalline nanoparticle catalyst of the present invention 10 is depicted. In such a material, each layer of a given crystallographic plane 11 (one such example being the {111} family of planes) is oriented with all equivalent planes throughout the entire nanostructure. In other instances, a polycrystalline catalyst may be preferred. FIG. 2 illustrates polycrystalline nanoparticle 20 where a plurality of crystal grains 21a, 21b, and 21c are contained within the volume of the nanoparticle 20. The plurality of crystal grains 21a, 21b, and 21c are separated by grain boundaries 22a, 22b, and 22c. Crystallographic planes 23a, 23b, and 23c are meant to be depicted as belonging to the same crystallographic family of planes (e.g., the {111} family of planes) and each is contained with a distinct grain (21a, 21b, and 21c, respectively). A feature of polycrystallinity is that—although the planes are crystallographically equivalent—the planes are not parallel to one another from one grain to another as they otherwise would be if the particle were single crystalline, as is shown in FIG. 1. While FIG. 2 illustrates three grains 21a, 21b, and 21c, it is contemplated in the context of the present invention that only two grains may be present or four, five, six, seven, eight, or more grains may be present in each nanostructure (e.g., each nanoparticle).

5. Catalyst Size

The nanostructured catalysts of the present invention can have at least one, at least two, or all three dimensions of 2 nanometers to 50 nanometers, preferably 5 nanometers to 40 nanometers, more preferably 10 nanometers to 30 nanometers, or most preferably 10 nanometers to 25 nanometers, and all ranges there between, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, and 49 nanometers. In another embodiment, the α-Fe₂O₃ nanostructures can be nanowires, nanoparticles, nanorods, nanotubes, tetrapods, nanocubes, or any other isotropic or anisotropic nanostructured material wherein the critical features sizes are within the above stated ranges. Various techniques may be employed to determine the size of any given nanostructure, often resulting in disparate values reported, each of which depends heavily on the technique used to make such a measurement. For example, the Fe₂O₃ particles reported by see Xiang et al. measured, on average, 57 nanometers when using x-ray diffraction (XRD) analysis; the same particles were reported as having a size of 98 nanometers when measured using transmission electron microscopy (TEM). TEM is the preferred method for measuring nanostructure size, considering the technique can be calibrated against the crystalline lattice of the material under investigation (which effectively serves as an internal measuring stick). By contrast, particle size as measured by XRD relies on the Scherrer equation, an empirical formula that correlates peak broadening in a diffractogram. Because effects other than size give rise to peak broadening, the Scherrer equation is only valid for nanoparticles no greater than 20 nm. Further, the Scherrer analysis measures grain size, which—considering the nanostructures may be polycrystalline—may be misleading toward the actual particle size (i.e., a nanostructure may have a size of 50 nanometers, but be comprised of polycrystalline grains, each of which has an average size of 5 nanometers; using the Scherrer analysis, one would be misled into believing that the average particle size was 5 nanometers, whereas TEM would reveal the true particle size of 50 nanometers). For these reasons, TEM—and not XRD—is the preferred method of determining particle size.

6. Support Material

In other non-limiting aspects of the present invention, the catalysts can be supported by a support material. The support material can be porous and have a high surface area. In some embodiments, the support includes an inorganic oxide, alpha, beta or theta alumina (Al₂O₃), activated Al₂O₃, silicon dioxide (SiO₂), tin oxide (SnO₂), titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₂O₄), lantheum oxide (La₂O₃), activated carbon, silica gel, zeolites, activated clays, silicon carbide (SiC), diatomaceous earth, magnesia, aluminosilicate, calcium aluminate, carbon nanotubes (CNT), boron nitride nanotubes (BNNT), or combinations thereof.

B. Methods for Making the α-Fe₂O₃ Catalyst

The catalysts of the present invention can be prepared through a staged thermal treatment process. The process can include obtaining a composition comprising precursors of iron and dopant(s) and/or promoter(s). The composition can be an aqueous solution or an organic solution where the iron precursor materials and optionally the dopants and/or promoters are solubilized therein. Ligands can also be included in the composition. The iron-containing precursor material can be an iron salt, an organometallic iron compound, an iron-containing complexed compound, or any other suitable precursor material, or mixtures or combinations thereof. Similarly, the dopant(s) and/or promoter(s) can be salts, organometallics, complexes, or other appropriate compounds, or mixtures or combinations thereof. Appropriate ligands may comprise organic compounds from the following classes: alkenes, alkynes, alcohols, aldehydes, carboxylic acids, phosphines, phosphonates, phosphonic acids, sulfates, sulfides, amines, or any other appropriate class of compound, or any combination or mixture thereof.

The composition can be initially subjected to an initial drying step to obtain crystalline material. The drying can be performed by subjecting the composition to a temperature of 80° C. to 200° C. for 30 minutes to 24 hours, preferably 90° C. to 180° C. This temperature and time range can be modified as desired. Once dried, the material can optionally be rinsed or washed with, for example, an aqueous solution or an organic solution.

The dried material can then be subjected to a staged calcination process. The first stage can include thermally heating the material at 400° C. to 600° C. (or any range there between such as 410° C., 420° C., 430° C., 440° C., 450° C., 460° C., 470° C., 480° C., 490° C., 500° C., 510° C., 520° C., 530° C., 540° C., 550° C., 560° C., 570° C., 580° C., 590° C., and 595° C.) in the presence of an oxygen source for 30 minutes to 4 hours (or any range there between, including 1, 2, and 3 hours). The second stage can include subjecting the material to a temperature of 750° C. to 850° C. (or any range there between, including 760° C., 770° C., 780° C., 790° C., 800° C., 810° C., 820° C., 830° C., 840° C., and 845° C.) for 30 minutes to 4 hours (or any range there between, including 1, 2, and 3 hours) to obtain the nanostructured α-phase hematite catalyst of the present invention. The second stage thermal treating step can immediately follow the first stage thermal treating step (e.g., by incrementally increasing the temperature to 750° C. to 850° C. such as by 10° C./minute). Alternatively, the material can be cooled after the first stage and then subjected to the second stage thermal treating step. In either instance, the resulting product is an α-Fe₂O₃ catalyst of the present invention having the size and composition described throughout this specification.

C. Methods of Using the α-Fe₂O₃ Catalyst

The catalysts of the present invention can be used in hydrocarbon dehydrogenation reactions, with dehydrogenation of ethylbenzene to styrene being a preferred reaction. The process can include contacting a feed stream of dehydrogenation-susceptible hydrocarbons with any of the catalysts described throughout the specification under desired reaction conditions. The feed stream can also include a carrier gas that does not negatively affect the reaction. Examples of such carrier gases include carbon dioxide, argon, or nitrogen. In a preferred embodiment, the reaction is dehydrogenation of ethylbenzene to styrene, wherein the catalyst is in contact with a reactant feed that includes ethylbenzene at a reaction temperature of 500° C. to 700° C. or any range there between, including 505° C., 510° C., 515° C., 520° C., 525° C., 530° C., 535° C., 540° C., 545° C., 550° C., 555° C., 560° C., 565° C., 570° C., 575° C., 580° C., 585° C., 590° C., 595° C., 600° C., 605° C., 610° C., 615° C., 620° C., 625° C., 630° C., 635° C., 640° C., 645° C., 650° C., 655° C., 660° C., 665° C., 670° C., 675° C., 680° C., 685° C., 690° C., and 695° C. Pressure used in the reaction can range from about 0.01 MPa to about 2.0 MPa. The gas hourly space velocity (GHSV) of the reactant feed can range from 100 h⁻¹ to 100,000 h⁻¹ or more.

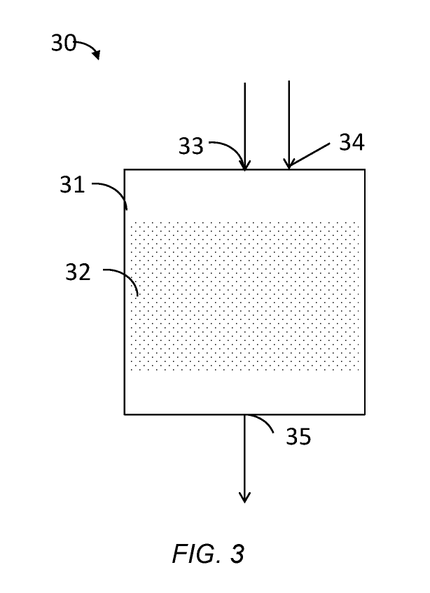

Referring to FIG. 3, a schematic of a system (30) for the production of a dehydrogenated hydrocarbon (e.g., styrene) is depicted. System (30) may include a continuous flow reactor (31) and the supported or unsupported α-Fe₂O₃ catalyst of the present invention (32). A reactant stream that includes ethylbenzene can enter the continuous flow reactor (31) via a feed inlet (33). In one embodiment, the reactants can be provided to the continuous flow reactor (31) such that the reactants are heated to a temperature between ambient (i.e., room temperature) and the reaction temperature prior to contacting the catalyst (32). In another embodiment, the catalytic material and the reactant feed are each heated to the approximately the same temperature. In some instances, the catalyst (32) may be layered in the continuous flow reactor (31). In other instances, the catalyst may be fed to the reactor while in contact to the reactant mixture in a fluidized bed configuration. In such an embodiment, the catalyst may be pre-mixed with the reactant stream prior to entering the reactor (31) or it may be introduced through a second inlet stream (34) and brought into contact with the reactant feed immediately thereafter. Contact of the reactant mixture with the catalyst (32) produces a product stream (for example, styrene and other hydrocarbon by-products). The product stream can exit the continuous flow reactor (31) via product outlet (35).

The resulting dehydrogenated hydrocarbons produced from the systems of the invention can be separated from any by-products using a variety of known gas/liquid separation techniques such as distillation, absorption, membranes, etc., to produce a purified dehydrogenated hydrocarbon product stream. The dehydrogenated products can then be used in additional downstream reaction schemes to create additional products. By way of example, styrene can be used to produce a variety of polymers such as latex, synthetic rubber, and polystyrene resins.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### Prophetic Method of Making a Catalyst of the Present Invention

A solution containing 0.01 to 5 M iron chloride (FeCl₃) and 0 to 5 M KNO₃ and 0 to 2 M antimony chloride and/or 0 to 2 M tin (IV) chloride (SnCl₄) and 0.1 to 10 M NaNO₃ will be mixed with fluorine or antimony doped tin oxide particles having an average particle size of 10-100 nm. The doped tin oxide particles will act as the substrate. Nanostructured iron oxyhydroxides (FeOOH) (yellow) film will be formed after heating the solution at 90° C. to 110° C. for 2 to 10 hours. Subsequently sintering 1 to 3 hours at 550° C. will result in a hematite-based nanostructure. Further heat treatment will be carried out at 800° C. for 0.5 to 2 hours to obtain a sinter-resistant hematite based catalyst of the present invention.

The amounts of the aforementioned materials used in this preparation process can be modified as desired. Further, the specific materials noted in the above paragraph can be modified or replaced with other materials described throughout this application to prepare all of the various nanostructured α-phase hematite-based catalysts of the present invention.

### Example 2

### Prophetic Method of Testing a Catalyst of the Present Invention

The following procedure can be used to determine the ability of the α-phase hematite-based catalysts of the present invention to catalyze a dehydrogenation of hydrocarbon reaction. The α-hematite based catalyst will be loaded into a reactor for ethylbenzene dehydrogenation reaction. The reaction will be carried out at near atmospheric pressure at temperature ranged 550 to 650° C. with a water to ethylbenzene ratio of 1:1 to 2:1 (wt. %). The catalyst will be tested for aging effect and will be compared with commercial available product(s). The surface of post-testing samples will be analyzed.

1. A nanostructured α-phase hematite-based catalyst capable of catalyzing a dehydrogenation of hydrocarbon reaction, the catalyst comprising an α-Fe₂O₃ crystalline phase and a dopant(s) and/or a promoter(s), wherein the catalyst has a size of 2 nanometers to 50 nanometers and does not include a γ-Fe₂O₃ crystalline phase.

2. The nanostructured α-phase hematite-based catalyst of claim 1, wherein the catalyst is a single- or poly-crystalline α-Fe₂O₃ phase catalyst.

3. The nanostructured α-phase hematite-based catalyst of claim 1, wherein the catalyst does not include a magnetite (Fe₃O₄) crystalline phase.

4. The nanostructured α-phase hematite-based catalyst of claim 1, wherein the catalyst has a general structure of:
[FeₓA_{y}B_{z}]Oₙ,
wherein A is a dopant, B is a promoter, x is 1−(y+z), 0≦y≦0.10, 0≦z≦0.30, n is determined by valence requirements of Fe and A and/or B, with the proviso that y or z is greater than 0.

5. The nanostructured α-phase hematite-based catalyst of claim 1, wherein the dopant(s) and/or promoter(s) is/are comprised in the lattice structure of the α-Fe₂O₃ crystalline phase.

6. The nanostructured α-phase hematite-based catalyst of claim 1, having a size of 2 nanometers to 40 nanometers, 5 nanometers to 30 nanometers, or 10 nanometers to 25 nanometers.

7. The nanostructured α-phase hematite-based catalyst of claim 1, wherein the catalyst comprises a dopant, the dopant comprising antimony (Sb), arsenic (As), bismuth (Bi), silicon (Si), germanium (Ge), tin (Sn), lead (Pb), aluminium (Al), gallium (Ga), indium (In), thallium (Tl), titanium (Ti), vanadium (V), chromium (Cr), molybdeum (Mo), manganese (Mn), platinum (Pt), lanthanum (La), cerium (Ce), or any combination, or oxide, or alloy thereof.

8. The nanostructured α-phase hematite-based catalyst of claim 1, wherein the catalyst comprises a promoter, the promoter comprising an alkali metal, an alkali earth metal, a transition metal, or a lanthanoid, or any combination, or oxide, or alloy thereof selected from Lithium (Li), Sodium (Na), Potassium (K), Rubidium (Rb), Cesium (Cs), Beryllium (Be), Magnesium (Mg), Calcium (Ca), Strontium (Sr), Barium (Ba), Scandium (Sc), Yttrium (Y), Zirconium (Zr), Hafnium (Hf), Vanadium (V), Niobium (Nb), Tantalum (Ta), Chromium (Cr), Molybdenum (Mo), Tungsten (W), Manganese (Mn), Technetium (Tc), Rhenium (Re), Ruthenium (Ru), Osmium (Os), Cobalt (Co), Rhodium (Rh), Iridium (Ir), Nickel (Ni), Palladium (Pd), Platinum (Pt), Copper (Cu), Silver (Ag), Gold (Au), Zinc (Zn), Cadmium (Cd), Mercury (Hg), Aluminum (Al), Gallium (Ga), Indium (In), Thallium (Tl), Germanium (Ge), Lead (Pb), Lanthanum (La), Cerium (Ce) Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (TM), Ytterbium (Yb), or Lutetium (Lu).

9. The nanostructured α-hematite-based catalyst of claim 1, wherein the catalyst is the product of a hydrothermal reaction of (i) a composition comprising precursors of iron and the dopant and/or promoter with (ii) thermal treatment, wherein the thermal treatment comprises subjecting the composition to a temperature of 80° C. to 200° C. for 30 minutes to 24 hours, followed by a temperature of 400° C. to 600° C. for 30 minutes to 4 hours, followed by a temperature of 750° C. to 850° C. for 30 minutes to 4 hours.

10. The nanostructured α-hematite-based catalyst of claim 1, wherein the catalyst is a bulk catalyst having an average nanostructure size of 2 nanometers to 50 nanometers, 2 nanometers to 40 nanometers, 5 nanometers to 30 nanometers, or 10 nanometers to 25 nanometers.

11. The nanostructured α-hematite-based catalyst of claim 1, wherein the catalyst is sinter and/or coke resistant during use.

12. The nanostructured α-hematite-based catalyst of claim 1, wherein the dehydrogenation of hydrocarbon reaction is dehydrogenation of ethylbenzene to styrene.

13. The nanostructured α-hematite-based catalyst of claim 12, wherein the catalyst is in contact with a reactant feed that includes ethylbenzene.

14. The nanostructured α-hematite-based catalyst of claim 13, wherein the reactant feed has a temperature of 500° C. to 700° C.

15. A method of dehydrogenating a hydrocarbon, the method comprising contacting a reactant feed that includes a hydrocarbon with any one of the nanostructured α-phase hematite-based catalysts of claim 1 under conditions sufficient to dehydrogenate the hydrocarbon.

16. The method of claim 15, wherein the hydrocarbon in the reactant feed is ethylbenzene, and wherein the ethylbenzene is dehydrogenated to styrene.

17. The method of claim 16, wherein the reactant feed has a temperature of 500° C. to 700° C.

18. A method of making a nanostructured α-phase hematite-based catalyst of claim 1, the method comprising:
(a) obtaining an aqueous solution comprising an iron containing precursor material and a dopant(s) and/or a promoter(s), wherein each of the precursor material and the dopant(s) and/or promoter(s) are solubilized in the aqueous solution;
(b) heating the aqueous solution to obtain a crystalline material comprising iron and dopant(s) and/or promoter(s), wherein the crystalline material has an average size of 2 nanometers to 50 nanometers; and
(c) subjecting the crystalline material from step (b) to a temperature of 400° C. to 600° C. for 30 minutes to 4 hours in the presence of an oxygen source followed by a temperature of 750° C. to 850° C. for 30 minutes to 4 hours to obtain the nanostructured α-phase hematite catalyst of any one of claims 1 to 14.

19. The method of claim 18, wherein the iron containing precursor material is an iron salt.

20. The method of claim 18, wherein heating step (b) comprises heating the aqueous solution to 80 to 200° C., preferably 90° C. to 180° C., for 30 minutes to 24 hours.
